(19) United States
(12) Patent Application Publication
(10) Pub. No.: US 2025/0082963 A1
(43) Pub. Date: Mar. 13, 2025

(54) MEDICAL RADIATION SHIELDING DEVICE

(71) Applicant: KANGWON NATIONAL UNIVERSITY HOSPITAL, Gangwon-do (KR)

(72) Inventors: Jun Bong SHIN, Seoul (KR); Minsik LEE, Chuncheon-si (KR); Nuri Hyun JUNG, Chuncheon-si (KR)

(73) Assignee: KANGWON NATIONAL UNIVERSITY HOSPITAL, Gangwon-do (KR)

(21) Appl. No.: 18/956,540

(22) Filed: Nov. 22, 2024

Related U.S. Application Data
(63) Continuation of application No. PCT/KR2023/006878, filed on May 19, 2023.

(30) Foreign Application Priority Data
May 23, 2022 (KR) 10-2022-0062627

(51) Int. Cl. A61N 5/10

(52) U.S. Cl. CPC A61N 5/10; A61N 2005/1094

(57) ABSTRACT

The present disclosure relates to a medical radiation shielding device including a bed irradiated with radiation from an outside, a shielding cover that is provided to be movable in a longitudinal direction of the bed and forms a radiation shielding space for shielding the radiation in the bed, a shielding seat that is provided to be movable in the longitudinal direction of the bed, is disposed between the bed and the shielding cover, and shields the radiation radiated to the radiation shielding space, and a pair of holders to which the shielding cover and the shielding seat are independently coupled to be movable in the longitudinal direction of the bed.

### CROSS-REFERENCE TO RELATED APPLICATIONS

The present application is a continuation of International Patent Application No. PCT/KR2023/006878, filed on May 19, 2023, which is based upon and claims the benefit of priority to Korean Patent Application No. 10-2022-0062627 filed on May 23, 2022. The disclosures of the above-listed applications are hereby incorporated by reference herein in their entirety.

### BACKGROUND

Embodiments of the present disclosure described herein relate to a medical radiation shielding device.

In recent years, with the rapid development of society, the incidence of cancers such as gastric cancers, liver cancers, colon cancers, and breast cancers is rapidly increasing, and accordingly, various diagnosis and treatment technologies are required. Examples of the treatment technology include chemotherapy (anti-cancer drugs) and invasive surgical therapy, but non-invasive radiation therapy is widely used.

In radiation therapy, therapeutic radiation irradiation devices and diagnostic radiation irradiation devices are widely used.

The therapeutic radiation irradiation device serves to delay, prevent, or extinguish growth of a tumor tissue included in a target tissue of a human body by radiating radiation to the target tissue and damaging or destroying the target tissue. Representative examples of the therapeutic radiation irradiation device include a linear accelerator (LINAC).

The diagnostic radiation irradiation device serves to capture an image of the target tissue by radiating the radiation to the target tissue of the human body. Examples of the diagnostic radiation irradiation device include a computerized tomography (CT) device, an X-ray device, and the like.

In recent years, a radiation treatment system, in which the target tissue is photographed by the CT device or the X-ray device, and when the tumor tissue is included in the target tissue, the target tissue is destroyed in real time in connection with the LINAC, is widely used.

However, a radiation treatment system according to the related art has a problem in that the radiation is radiated to a surrounding tissue of the target tissue, and thus the surrounding tissue is destroyed.

To solve this problem, protective clothing (e.g., an apron or the like) for shielding the radiation radiated to the surrounding tissue of the target tissue is provided. Adult patients have no resistance to wearing the protective clothing, but pediatric patients feel uncomfortable in wearing the protective clothing or do not wear the protective clothing.

Furthermore, in the case of the protective clothing, it is best to selectively set a radiation shielding space of the protective clothing in consideration of an anatomical structure of the patient, the target tissue, and a magnitude of radiation energy. However, most protective clothing is designed to protect the entire human body, and thus selective shielding of the radiation is impossible or customization for each of patients is difficult and costs are generated. Thus, it is difficult to apply the protective clothing to radiation diagnosis and treatment.

### SUMMARY

Embodiments of the present disclosure provide a radiation shielding device that may easily adjust and set a position of a radiation shielding space.

The aspects of the present disclosure are not limited to the aspects described above, and those skilled in the art will clearly understand other aspects not described from the following description.

According to an embodiment, a medical radiation shielding device includes a bed irradiated with radiation from an outside, a shielding cover that is provided to be movable in a longitudinal direction of the bed and forms a radiation shielding space for shielding the radiation in the bed, a shielding seat that is provided to be movable in the longitudinal direction of the bed, is disposed between the bed and the shielding cover, and shields the radiation radiated to the radiation shielding space, and a pair of holders to which the shielding cover and the shielding seat are independently coupled to be movable in the longitudinal direction of the bed.

Further, each of the holders may include a holder body, a cover track that is provided on an upper surface of the holder body in the longitudinal direction of the bed and guides movement of the shielding cover, and a seat track that is provided on an inner surface of the holder body in the longitudinal direction of the bed and guides movement of the shielding seat.

Further, the medical radiation shielding device may further include a cover slider coupled to an end portion of the shielding cover and moving along the cover track and a seat slider coupled to an end portion of the shielding seat and moving along the seat track.

Further, the medical radiation shielding device may further include a stopper that is detachably provided in the pair of holders and limits the movement of the shielding cover and the shielding seat.

Further, the stopper may include a plurality of stopper grooves provided in the pair of holders at intervals in the longitudinal direction of the bed, a stopper bar crossing the pair of holders and held on the pair of holders, and a fastening bolt that selectively fastens the stopper bar to one of the plurality of stopper grooves.

Further, the shielding cover may be made of a transparent or opaque material.

Further, the shielding cover and the shielding seat may include a first shielding cover and a first shielding seat that forms a first radiation shielding space in one area of the bed and a second shielding cover and a second shielding seat that forms a second radiation shielding space in the other area of the bed, and at least one of the first shielding cover and the second shielding cover may be made of a transparent material.

Other detailed matters of the present disclosure are included in the detailed description and the accompanying drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating a medical radiation shielding device according to an embodiment of the present disclosure;

FIG. 2 is an exploded perspective view illustrating the medical radiation shielding device according to the embodiment of the present disclosure;

FIG. 3 is a cross-sectional view illustrating the medical radiation shielding device according to the embodiment of the present disclosure;

FIG. 4 is a perspective view illustrating a state in which a medical radiation shielding device according to the embodiment of the present disclosure is applied to a radiation irradiation device; and

FIGS. 5 to 7 are schematic views illustrating a process of adjusting and setting a radiation shielding space by the medical radiation shielding device according to the embodiment of the present disclosure.

### DETAILED DESCRIPTION

Advantages and features of the present disclosure and a method of achieving the advantages and the features will become apparent with reference to embodiments described below in detail together with the accompanying drawings. However, the inventive concept is not limited to the embodiments described below but may be implemented in various forms, and the present embodiments merely make the disclosure of the inventive concept complete and are provided to completely inform the scope of the inventive concept to those skilled in the art to which the inventive concept belongs, and the inventive concept is merely defined by the scope of the appended claims.

Terms used in the present specification are intended to describe an embodiment and are not intended to limit the inventive concept. In the specification, a singular form also includes a plural form unless specifically mentioned in a phrase. The terms “comprises” and/or “comprising” used in the specification do not exclude the presence or addition of one or more other components other than the mentioned components. Throughout the specification, the same reference numerals refer to the same components, and the term “and/or” includes each and all combinations of one or more of components mentioned. Although “first,” “second,” and the like are used to describe various components, it is apparent that these components are not limited by these terms. These terms are only used to distinguish one component from another component. Thus, it is apparent that a first component mentioned below may be a second component within the technical spirit of the inventive concept.

Unless otherwise defined, all the terms (including technical and scientific terms) used herein may be used as meanings that may be commonly understood by those skilled in the art to which the inventive concept belongs. Further, terms defined in a commonly used dictionary are not interpreted ideally or excessively unless explicitly and specifically defined.

Spatially relative terms such as “below”, “beneath”, “lower”, “above”, and “upper” may be used to easily describe a correlation between a first component and a second component as illustrated in the drawings. The spatially relative terms should be understood as terms including different directions of components during use or operation in addition to directions illustrated in the drawings. For example, when the first component illustrated in the drawings is turned over, the first component described to be located “below” or “beneath” the second component may be placed “above” the second component. Thus, the exemplary term “below” may include both downward and upward directions. The components may be oriented in other directions, and accordingly, the spatially relative terms may be interpreted according to the orientation.

Hereinafter, embodiments of the inventive concept will be described in detail with reference to the accompanying drawings.

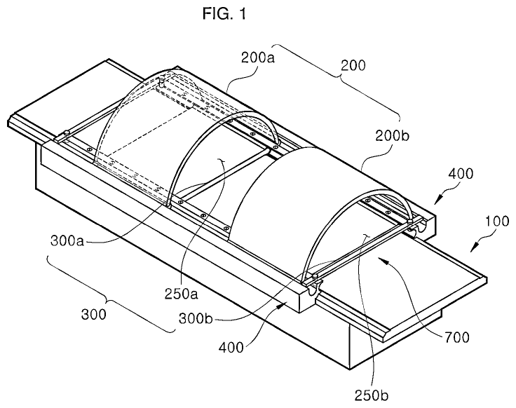

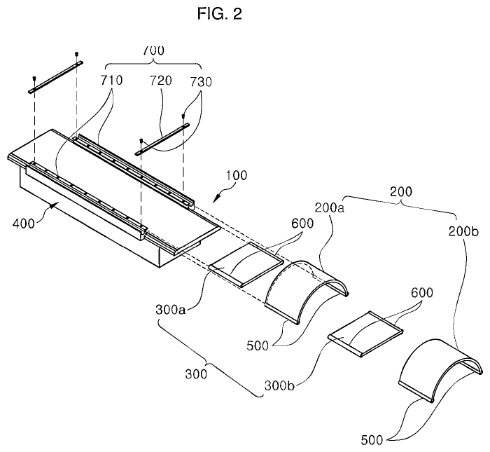

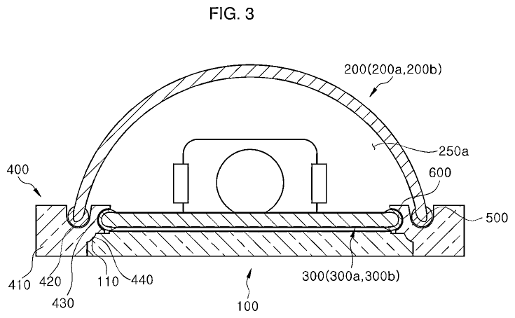

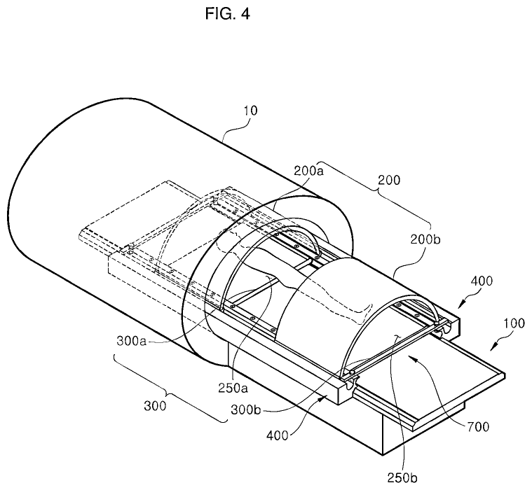

FIG. 1 is a perspective view illustrating a medical radiation shielding device according to an embodiment of the present disclosure, FIG. 2 is an exploded perspective view illustrating the medical radiation shielding device according to the embodiment of the present disclosure, FIG. 3 is a cross-sectional view illustrating the medical radiation shielding device according to the embodiment of the present disclosure, and FIG. 4 is a perspective view illustrating a state in which a medical radiation shielding device according to the embodiment of the present disclosure is applied to a radiation irradiation device.

As illustrated in FIGS. 1 to 3, a medical radiation shielding device according to an embodiment of the present disclosure includes a bed 100, a shielding cover 200, a shielding seat 300, and a pair of holders 400.

The bed 100 is irradiated with radiation from the outside, and a body 1 of an examinee or a person to be treated may be laid on the bed 100. Here, the radiation radiated to the bed 100 may be therapeutic radiation for treating a target tissue of the body 1 or diagnostic radiation for diagnosing and photographing the target tissue of the body 1.

As illustrated in FIG. 4, the bed 100 may be provided to enter a radiation irradiation device for radiating the therapeutic radiation or the diagnostic radiation. For example, the bed 100 may be provided with a wheel that may manually enter the radiation irradiation device. Further, the bed 100 may be provided with an actuator that may automatically enter the radiation irradiation device.

The bed 100 may be formed in a rectangular shape as a whole.

In the embodiment, a matching groove 110 may be formed around the bed 100. Here, the matching groove 110 may be recessed around the bed 100 in a longitudinal direction of the bed 100. A matching protrusion 440 of the holder 400, which will be described later, may be matched to the matching groove 110. Thus, the holder 400 may be firmly seated around the bed 100.

In the embodiment, the holder 400 may be held on an upper surface of the bed 100.

The shielding cover 200 is provided to be movable in the longitudinal direction of the bed 100 and forms radiation shielding spaces 250a and 250b for shielding radiation in the bed 100. The body 1 may be accommodated in the radiation shielding spaces 250a and 250b.

For example, the shielding cover 200 may have an arc-shaped cross-sectional shape.

The shielding seat 300 is provided to be movable in the longitudinal direction of the bed 100, is disposed between the bed 100 and the shielding cover 200, and shields the radiation radiated into the radiation shielding spaces 250a and 250b. For example, the shielding seat 300 may have a plate-shaped cross-sectional shape.

In this way, the shielding cover 200 and the shielding seat 300 may serve to form the radiation shielding spaces 250a and 250b for shielding the radiation in the bed 100, the shielding cover 200 may shield radiation radiated from the outside of the shielding cover 200 to the radiation shielding spaces 250a and 250b, and the shielding seat 300 may shield radiation irradiated from the outside of the bed 100 to the radiation shielding spaces 250a and 250b.

Furthermore, since both the shielding cover 200 and the shielding seat 300 are provided to be movable in the longitudinal direction of the bed 100, locations of the radiation shielding spaces 250a and 250b in which the shielding cover 200 and the shielding seat 300 are formed in the bed 100 may be easily adjusted and set by adjusting locations of the shielding cover 200 and the shielding seat 300 in the bed 100.

In the embodiment, the shielding cover 200 may be formed of a transparent or opaque material. Here, the shielding cover 200 may include a first shielding cover 200a in which one area of the body 1 of the examinee or the person to be treated is accommodated and a second shielding cover 200b in which the other area of the body 1 is accommodated, the first shielding cover 200a may be formed of a transparent material, and the second shielding cover 200b may be formed of an opaque material. In this way, when the first shielding cover 200a is made of a transparent material, an inspector or operator may identify a state of the first radiation shielding space 250a through the first shielding cover 200a. Further, when a face of the examinee or the person to be treated is located in the first radiation shielding space 250a, the examinee or the person to be treated may visually identify a surrounding situation of the first shielding cover 200a through the first shielding cover 200a.

In the embodiment, the shielding cover 200 and the shielding seat 300 may include the first shielding cover 200a and a first shielding seat 300a for forming the first radiation shielding space 250a in one area of the bed 100 and the second shielding cover 200b and a second shielding seat 300b for forming the second radiation shielding space 250b in the other area of the bed 100, the first shielding cover 200a may be formed of a transparent material, and the second shielding cover 200b may be formed of an opaque material. However, the present disclosure is not limited thereto, and both the first shielding cover 200a and the second shielding cover 200b may be formed of a transparent material. Further, the first shielding cover 200a may be formed of an opaque material, and the second shielding cover 200b may be formed of a transparent material.

The shielding cover 200 and the shielding seat 300 are independently coupled to the pair of holders 400 to be movable in the longitudinal direction of the bed 100. Thus, since a movement path of the shielding cover 200 and a movement path of the shielding seat 300 do not overlap each other in the pair of holders 400, the shielding cover 200 and the shielding seat 300 may independently move in the pair of holders 400.

As another embodiment, although not illustrated, the pair of holders 400 may be held on the upper surface of the bed 100.

The holder 400 may include a holder body 410, a cover track 420, and a seat track 430.

The holder body 410 is a basic body of the holder 400, has a block shape having a predetermined length, and is attached to and detached along both edges of the bed 100. The cover track 420 may be formed on an upper surface of the holder body 410, and the seat track 430 may be formed on an inner surface of the holder body 410.

The cover track 420 is provided on the upper surface of the holder body 410 in the longitudinal direction of the bed 100 and guides movement of the shielding cover 200. Here, a cover slider 500 may be provided at an end portion of the shielding cover 200.

The cover slider 500 may be coupled to the end portion of the shielding cover 200 and may move along the cover track 420. For example, the cover slider 500 may have an annular cross-sectional shape, and in this case, the cover track 420 is recessed in the holder body 410 in a cross-sectional shape corresponding to the cover slider 500 so that the cover slider 500 may move in a sliding manner.

The seat track 430 is provided on the inner surface of the holder body 410 in the longitudinal direction of the bed 100 and guides movement of the shielding seat 300. Here, a seat slider 600 may be provided at an end portion of the shielding seat 300.

The seat slider 600 may be coupled to the end portion of the shielding seat 300 and may move along the seat track 430. For example, the seat slider 600 may have an annular cross-sectional shape, and in this case, the seat track 430 is recessed in the holder body 410 in a cross-sectional shape corresponding to the seat slider 600 so that the seat slider 600 may move in a sliding manner.

Meanwhile, the medical radiation shielding device according to the embodiment of the present disclosure may further include a stopper 700.

The stopper 700 is detachably provided on the pair of holders 400 and serves to limit the movement of the shielding cover 200 and the shielding seat 300. The stopper 700 may include a plurality of stopper grooves 710, a stopper bar 720, and a fastening bolt 730.

The plurality of stopper grooves 710 may be provided in the pair of holders 400 at intervals in the longitudinal direction of the bed 100.

Here, the plurality of stopper grooves 710 may communicate with the seat track 430 of the holder 400. Thus, the fastening bolt 730 fastened to the stopper groove 710 may protrude to the seat track 430, may be caught by the end portion of the shielding seat 300 that moves along the corresponding seat track 430, and may limit the movement of the shielding seat 300 that moves along the seat track 430.

For example, when the fastening bolt 730 is fastened to one of the plurality of stopper grooves 710, the fastening bolt 730 fastened to the corresponding stopper groove 710 may protrude to one side of the seat track 430, may be caught by the end portion of the shielding seat 300 that moves along the corresponding seat track 430, and may limit an one-way movement path of the shielding seat 300 that moves along the corresponding seat track 430. In other words, a forward movement path or a rearward movement path of the shielding seat 300 is limited.

Further, when the fastening bolts 730 are fastened to one pair of the plurality of stopper grooves 710, the fastening bolts 730 fastened to the one pair of stopper grooves 710 may protrude to both sides of the seat track 430 and may limit a two-way movement path of the shielding seat 300 that moves along the corresponding seat track 430. In other words, the forward/backward movement path of the shielding seat 300 is limited.

The stopper bar 720 may cross the pair of holders 400 and may be held on the pair of holders 400. Here, the stopper bar 720 may serve to maintain a gap between the pair of holders 400.

Here, an end portion of the stopper bar 720 may cover the cover track 420 of the holder 400. Thus, the end portion of the stopper bar 720 that covers the corresponding cover track 420 may be caught by the shielding cover 200 that moves along the corresponding cover track 420 and may limit the movement of the shielding cover 200 that moves along the corresponding cover track 420.

For example, when the end portion of the one stopper bar 720 covers one side of the cover track 420 of the holder 400, the end portion of the one stopper bar 720 may be caught by the shielding cover 200 that moves along the corresponding cover track 420 and may limit the one-way movement path of the shielding cover 200 that moves along the corresponding cover track 420. In other words, a forward movement path or a rearward movement path of the shielding cover 200 is limited.

Further, when end portions of the pair of stopper bars 720 cover both sides of the cover track 420 of the holder 400, the end portions of the pair of stopper bars 720 may be caught by the shielding cover 200 that moves along the corresponding cover track 420 and may limit a two-way movement path of the shielding cover 200 that moves along the corresponding cover track 420. In other words, the forward/backward movement path of the shielding cover 200 is limited.

The fastening bolt 730 serves to selectively fasten the stopper bar 720 to any one of the plurality of stopper grooves 710.

Meanwhile, the stopper 700 may have only the stopper bar 720, and in this case, the stopper bar 720 serves to maintain a gap between the pair of holders 400.

Hereinafter, a process of adjusting and setting the radiation shielding spaces 250a and 250b by the medical radiation shielding device according to the embodiment of the present disclosure will be described.

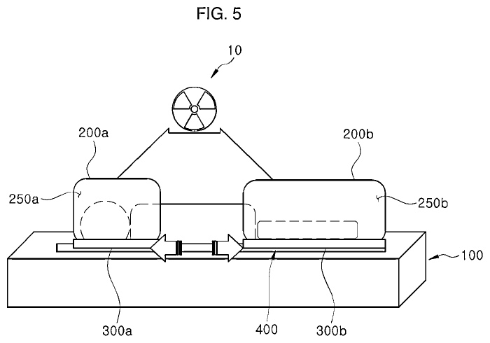

FIGS. 5 to 7 are schematic views illustrating a process of adjusting and setting the radiation shielding spaces 250a and 250b by the medical radiation shielding device according to the embodiment of the present disclosure.

As illustrated in FIG. 5, when a target tissue to which the radiation irradiation device radiates radiation is an abdomen of the body 1 of the examinee or the person to be treated, the first shielding cover 200a and the first shielding seat 300a, which are independently provided in the holder 400 to be movable in the longitudinal direction of the bed 100, are moved to the face and a chest of the body 1 to form the first radiation shielding space 250a, and the second shielding cover 200b and the second shielding seat 300b, which are independently provided in the holder 400 to be movable in the longitudinal direction of the bed 100, are moved to legs of the body 1 to form the second radiation shielding space 250b. In this case, as the first shielding cover 200a is made of a transparent material, the inspector or operator may identify the state of the first radiation shielding space 250a through the first shielding cover 200a. Further, when the face of the examinee or the person to be treated is located in the first radiation shielding space 250a, the examinee or the person to be treated may visually identify the surrounding situation of the first shielding cover 200a through the first shielding cover 200a.

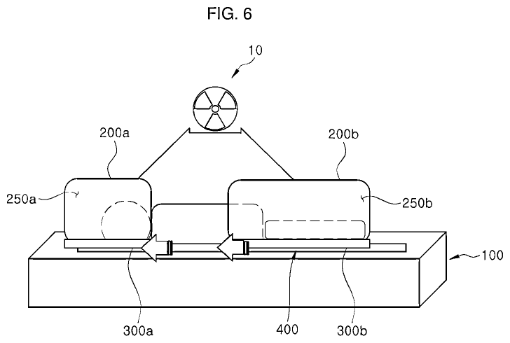

As illustrated in FIG. 6, when a target tissue to which the radiation irradiation device radiates radiation is the chest of the body 1, the first shielding cover 200a and the first shielding seat 300a, which are independently provided in the holder 400 to be movable in the longitudinal direction of the bed 100, are moved to the face of the body 1 to form the first radiation shielding space 250a, and the second shielding cover 200b and the second shielding seat 300b, which are independently provided in the holder 400 to be movable in the longitudinal direction of the bed 100, are moved to the abdomen and the legs of the body 1 to form the second radiation shielding space 250b. In this case, as the first shielding cover 200a is made of a transparent material, the inspector or operator may identify the state of the first radiation shielding space 250a through the first shielding cover 200a. Further, when the face of the examinee or the person to be treated is located in the first radiation shielding space 250a, the examinee or the person to be treated may visually identify the surrounding situation of the first shielding cover 200a through the first shielding cover 200a.

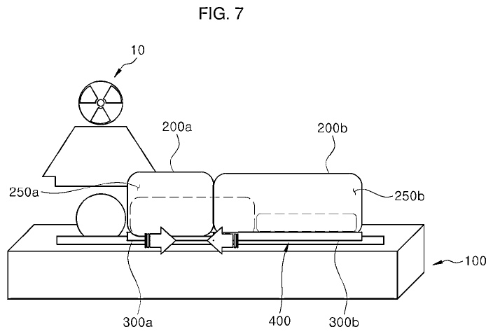

As illustrated in FIG. 7, when a target tissue to which the radiation irradiation device radiates radiation is the face of the body 1, the first shielding cover 200a and the first shielding seat 300a, which are independently provided in the holder 400 to be movable in the longitudinal direction of the bed 100, are moved to the chest of the body 1 to form the first radiation shielding space 250a, and the second shielding cover 200b and the second shielding seat 300b, which are independently provided in the holder 400 to be movable in the longitudinal direction of the bed 100, are moved to the abdomen and the legs of the body 1 to form the second radiation shielding space 250b.

The medical radiation shielding device according to the embodiment of the present disclosure may easily adjust and set the location of the radiation shielding space.

Hereinabove, the embodiments of the present disclosure have been described with reference to the accompanying drawings. However, those skilled in the art to which the present disclosure pertains may understand that the present disclosure may be implemented in other specific forms without changing the technical spirit or essential features thereof. Therefore, it should be understood that the embodiments described above are illustrative but not limiting in all aspects.

What is claimed is:

1. A medical radiation shielding device comprising:
a shielding cover provided to be movable in a longitudinal direction of a bed irradiated with radiation from an outside and configured to form a radiation shielding space for shielding the radiation in the bed;
a shielding seat provided to be movable in the longitudinal direction of the bed, disposed between the bed and the shielding cover, and configured to shield the radiation radiated to the radiation shielding space; and
a pair of holders which are provided in the bed and to which the shielding cover and the shielding seat are independently coupled to be movable in the longitudinal direction of the bed.

2. The medical radiation shielding device of claim 1, wherein each of the holders includes:
a holder body;
a cover track provided on an upper surface of the holder body in the longitudinal direction of the bed and configured to guide movement of the shielding cover; and
a seat track provided on an inner surface of the holder body in the longitudinal direction of the bed and configured to guide movement of the shielding seat.

3. The medical radiation shielding device of claim 2, further comprising:
a cover slider coupled to an end portion of the shielding cover and moving along the cover track; and
a seat slider coupled to an end portion of the shielding seat and moving along the seat track.

4. The medical radiation shielding device of claim 3, further comprising:
a stopper detachably provided in the pair of holders and configured to limit the movement of the shielding cover and the shielding seat.

5. The medical radiation shielding device of claim 4, wherein the stopper includes:
a plurality of stopper grooves provided in the pair of holders at intervals in the longitudinal direction of the bed;
a stopper bar crossing the pair of holders and held on the pair of holders; and
a fastening bolt configured to selectively fasten the stopper bar to one of the plurality of stopper grooves.

6. The medical radiation shielding device of claim 2, wherein the holder is detachably provided in the bed and further includes:
a matching groove formed around the bed; and
a matching protrusion provided in the holder and matched to the matching groove.

7. The medical radiation shielding device of claim 1, wherein the shielding cover is made of a transparent or opaque material.

8. The medical radiation shielding device of claim 1, wherein the shielding cover and the shielding seat include:
a first shielding cover and a first shielding seat configured to form a first radiation shielding space in one area of the bed; and
a second shielding cover and a second shielding seat configured to form a second radiation shielding space in the other area of the bed, and
wherein at least one of the first shielding cover and the second shielding cover is made of a transparent material.
